(19) United States
(12) Patent Application Publication
(10) Pub. No.: US 2008/0178879 A1
(43) Pub. Date: Jul. 31, 2008

(54) IMPELLER FOR A WEARABLE POSITIVE AIRWAY PRESSURE DEVICE

(75) Inventors: Stephen Keir ROBERTS, Ottawa (CA); Donald Carmon BRADLEY, Kanata (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, omitted

(73) Assignee: BRAEBON MEDICAL CORPORATION, Carp (CA)

(21) Appl. No.: 11/668,310

(22) Filed: Jan. 29, 2007

(51) Int. Cl. A61M 16/00

(52) U.S. Cl. 128/204.18

(57) ABSTRACT

A method for increasing output pressure of a blower unit in a Positive Airway Pressure (PAP) device, the blower unit having a blower rotatable about an axis of rotation. The method includes the steps of ingesting air into the blower unit, successively accelerating the ingested air in a direction substantially radial to the axis of rotation and a direction substantially parallel to the axis of rotation for generating a flow of compressed air; and exhausting the accelerated air from the blower unit. A PAP device with the improved blower generates increased air pressure compared to prior art devices or produces at least the same air pressure at a reduced size.

### FIELD OF THE INVENTION

The present invention relates generally to an impeller for positive air pressure devices. More particularly, the present invention relates to an impeller for wearable positive air pressure devices.

### BACKGROUND OF THE INVENTION

Obstructive sleep apnea (OSA) is a condition that affects an estimated 14 million Americans. The condition is caused by relaxation of the soft tissue in the palate during sleep, resulting in obstruction of the upper airway. OSA is characterized by a complete cessation of breathing during sleep for 10 or more seconds (apnea), or a reduction in breathing for 10 or more seconds causing a 4% or greater decrease in blood oxygen level (hypopnea). Individuals having five or more apneic or hypopneic events per hour are diagnosed as suffering from OSA. The obvious side effects of sleep apnea are daytime sleepiness and chronic fatigue. However, OSA is known to be a contributing factor in hypertension, heart disease, as well as other serious health conditions.

The most common treatment for OSA is positive pressure (above-ambient) applied at the patient's nose or mouth, or at both the nose and mouth. This creates what is known as a pneumatic splint, which prevents the closing of the airway that causes apnea and hypopnea. Treatment pressures typically range between 4 and 20 cm H₂O, depending primarily on the severity of the condition.

FIG. 1 is a schematic diagram of a conventional device used in positive airway pressure (PAP) therapy treatment. The PAP device 10 typically comprises an intake air filter 20; a blower 38 that compresses the treatment air via fan blades 22; a flow sensor 24 and a pressure sensor 28 connected to an exhaust tube 26. A controller 36 uses the electrical signals generated by the flow sensor 24 and the pressure sensor 28 to control the motor of the blower 38. The PAP device 10 receives power from the power supply 34, which can be an AC/DC converter connected to an AC mains supply, or to a battery pack. The power supply 34 can be positioned internally, externally, or a combination thereof, as in an external AC power supply with internal AC/DC voltage conversion. The PAP device 10 is connected to a tube, which carries the pressurized air to an interface. The interface is adapted to deliver the pressurized air to the patient's nares, mouth, or both, and is operatively coupled to the PAP device 10 by flexible tubing, typically measuring 6 feet or more in length.

The PAP device 10 may also include other components or features that are adapted to increase patient comfort, or that are used for diagnostic purposes. Examples of comfort enhancing features include: air humidifiers 32 and heaters 30 that are designed to prevent soreness of the airway and larynx, by providing cold humidification or heated humidification.

FIG. 2 is a block diagram of the controller 36 and electrical connections of a conventional PAP device. The main microcontroller 37 is connected to memory 48, a motor microcontroller 50, and analog to digital converter 52, an over current sensor 54, and a communications module 46. The memory 48 is used for storing operating data and the motor microcontroller 50 is used to directly control a motor of the blower 38. The analog to digital converter 52 is used to provide the digital signals for the flow sensor 24, pressure sensor 28 and the over current sensor 54. The communication module 46 allows external communication to the device. The power supply 34 supplies power to the controller 36. An on/off switch 40, visual indicators 42 such as an LCD, and an audible indicator 44 may also be connected to the main microcontroller 37. The device may have other switches connected that allow the user to control the device. Other features available for advanced clinical control include advanced pressure control through reduced expiratory pressure; automatic pressure adjustments; and data acquisition and data storage functions that are used to log system performance and patient compliance.

The usual operational configuration of the PAP device consists of the PAP device sitting on a night table beside the bed. However, recent versions of the PAP device now have the device configured to be worn on a patient's body. For example, the device disclosed in United States Patent Application Publication No. US 2006/0096596 A1—Occhialini et al., has the PAP device and power supply located on the patient's body.

There are three common means by which positive airway pressure treatment is administered. The physical arrangement for the three methods is the same, the only substantial difference being in the programming of the controller. The first method is known as a continuous positive airway pressure (CPAP). A CPAP device is designed to maintain a prescribed positive pressure in the patient's airway at all times. The second method is known as bi-level positive airway pressure. Bi-level is similar to CPAP, except that the pressure alternates between two prescribed levels: a higher pressure during inhalation, and a lower pressure during exhalation. The third method is automatic positive airway pressure (APAP) treatment. An APAP device monitors the patient's breathing, and adjusts the positive pressure in response to apneic and hypopneic events, or other abnormal breathing. Each of the above methods has been demonstrated to provide effective treatment of OSA. Subsequent references in this document to positive airway pressure (PAP) treatment or devices are intended to include any or all of the above methods.

Although positive airway pressure is known to be an effective treatment for OSA, only 50% of patients prescribed PAP treatment use their device regularly. According to patient studies, the primary reason for this lack of compliance is that patients find the devices cumbersome and uncomfortable to wear. The size, weight, and alternating current (AC) power requirement restrict the patient's freedom for travel with the device, and freedom for movement while in bed. During use, patient movement frequently causes the tubing to tug on the interface, which may wake the patient. Tugging on the interface may also cause improper fit of the device, resulting in loss of effectiveness of the treatment and increased noise of the device due to air leakage. The 6-foot hose that normally connects the PAP device to the patient interface may also restrict body movement. The above factors are all likely to cause frequent patient arousal during the night, and contribute to the low level of compliance of PAP treatment.

Commercially available positive airway pressure devices require either direct AC connection for power, or, for portable devices, a substantially sized battery pack. For example, the lightest and most compact bedside PAP device currently on the market has a total weight of 2.1 lb, which does not include a power source. The wearable PAP system disclosed by Occhialini et al. is a lightweight and portable blower system made up of small air pumps, and requires a power supply weighing 1.68 kg (3.7 lb). Another commercially available device, such as taught in U.S. Pat. No. 7,012,346 to Hoffman et al., provides airway pressure between 5 cm H₂O and 12 cm H₂O, and has a total weight of 4.7 lb, including the battery. In spite of the reduced size, increased portability, and quieter operation of modern devices compared to their predecessors, inconvenience and awkwardness remain significant contributors to low patient compliance. In order to increase the patient compliance and usage, PAP devices need to be further miniaturized, must be more lightweight, and consume even less power than current alternatives.

The most significant obstacle to making devices used in treatment of sleep-disordered breathing portable is the high power consumption of the blower unit, therefore requiring a large and heavy stored energy device (i.e. battery). Typically, the largest and heaviest component in a portable PAP treatment apparatus is the battery or batteries. Minimizing battery size and weight can only be achieved with significant improvements in the efficiency of the blower unit, thereby minimizing the power consumption.

FIG. 3a is a schematic representation of the rotating blower 38 of the conventional PAP device 10 shown in FIG. 1. The rotating blower 38 is used to continuously pressurize a flow of gas by means of a rotating impeller disc 60 contained within a housing 62. The impeller disc 60 generally consists of a hub 64 that is fixed to the shaft 66 of a motor 68. The impeller disc 60 has a number of blades 22 protruding in a direction that is generally perpendicular to the surface of the impeller disc 60, and parallel to the axis of rotation of the disc, as shown in FIG. 3b. The housing 62 is used to enclose the gas as it passes through the impeller disc 60, and guide it toward the exit of the machine through the exhaust tube 26. In the most general sense, the impeller increases the pressure of the fluid contained in the housing by imparting it with angular momentum.

Rotating blowers are most often classified as either axial or radial, with the classification describing the meridional direction of the gas flow path, particularly as it exits the impeller. With reference to FIGS. 3a and 3b, the axial direction (z) is parallel to the axis of rotation of the motor 68 and impeller disc 60, and the radial direction (r) is perpendicular to this axis. The meridional direction is defined as the projection of the flow path in the plane formed by the axial (z) and radial directions (r). It is sometimes also thought of as a circumferential averaging of the flow and/or geometric quantities. Use of this terminology allows for discussion of the blower geometry and flow direction without the need to consider the component of flow in the circumferential (θ) direction. It is well understood in the art that in an axial impeller, the component of the output fluid flow path in a substantially axial (z) direction is greater than the component of the output fluid flow path in a substantially radial (r) direction and vice-versa in a radial impeller. References to axial and radial directions throughout this description are based on the abovementioned definitions.

Examples of axial impellers include ducted fans and propellers, in which the flow through the machine is primarily axial, with negligible radial motion. Axial impellers are generally characterized by high flow rates and low discharge pressures for a given rotational speed. Radial impellers, such as squirrel-cage blowers and centrifugal pumps, are characterized by a through flow which is primarily radial, with no significant axial component. These machines are generally used when high discharge pressures and lower flow rates are required for a given rotational speed. A third, and somewhat less common, type is the mixed-flow blower, where the direction of the flow exiting the impeller has significant components in both the axial (z) and radial (r) directions. These machines are most appropriate in applications where the flow rate and discharge pressure are both moderate. At operating speeds typical of currently available brushless DC motors, the radial impeller provides the best efficiency for pressures and flow rates typical of PAP treatment devices.

For a given geometric design, the pressure developed by a rotating impeller is approximately proportional to the square of the velocity of the impeller at its outer periphery. Thus, the output pressure may be increased either by increasing the rotational speed of the impeller, or by increasing the impeller diameter. Heretofore, several attempts have been made to design impellers with improved efficiency based on the construction and geometry of the impeller blades or vanes.

U.S. Pat. No. 6,681,033 to Makinson et al. teaches an impeller having a plurality of impeller vanes molded over the permanent magnets of an electric motor rotor. The impeller vanes are arranged in an annular array on the face of a disc shaped rotor and in a plane substantially perpendicular to the plane of the disc. The impeller vanes have a curved profile. Makinson et al. teach an improved construction of the impeller to reduce the imbalances of the completed rotor product thereby improving the efficiency of the impeller.

U.S. Pat. No. 6,622,724 to Truitt et al. discloses an impeller having a plurality of impeller blades disposed on a face of the impeller body with an inlet area between each pair of adjacent blades being substantially equal to a corresponding outlet area for each pair of adjacent blades. Maintaining of the inlet area substantially equal to the outlet area is believed to provide a substantially constant pressure gas at the outlet, despite fluctuations in the flow rate typically encountered in a respiratory pressure support system. This is achieved by having the blades decrease in height as they extend radially outward from the hub.

Other methods for improving the efficiency of an impeller used in PAP devices include the use of two impellers as taught by Daly et al. in U.S. Pat. No. 6,910,483.

All of the impellers described above are radial impellers characterized by a through flow which is primarily radial, with no significant axial component. Although radial impellers provide the best performance for pressures and flow rates typical of PAP treatment devices, there is significant scope for improving the efficiency of impellers in order to achieve miniaturization of impellers needed for wearable PAP devices.

It is, therefore, desirable to provide a method and a PAP device with an improved impeller of smaller size that produces at least the same air pressure as prior art devices or an increased air pressure for a given size.

### SUMMARY OF THE INVENTION

It is an object of the present invention to obviate or mitigate at least one disadvantage of previous impellers for wearable PAP devices. This is achieved with an impeller, which successively accelerates the pumped air in substantially radial and axial directions. The term acceleration in a substantially radial direction as used in this specification means that a major portion of the angular acceleration occurs in a direction perpendicular to the axis of rotation of the impeller. The term acceleration in a substantially axial direction means that a major portion of the angular acceleration occurs in a direction parallel to the axis of rotation of the impeller.

In a first aspect, the invention provides a method for increasing output pressure of a blower unit in a PAP device, the blower unit having a blower rotatable about an axis of rotation. The method includes the steps of ingesting air into the blower unit, successively accelerating the ingested air in a direction substantially radial to the axis of rotation and a direction substantially parallel to the axis of rotation for generating a flow of compressed air; and exhausting the accelerated air from the blower unit.

In a second aspect, the present invention provides a rotary impeller for a blower unit in a PAP device. The impeller has an axis of rotation and includes a rotatable impeller body, and radial vanes connected to the impeller body for accelerating air in a substantially radial direction upon rotation of the impeller body, to generate a generally radial air flow. Each radial vane has a pair of end portions which are, relative to the direction of the air flow, a leading portion with a leading edge and a trailing portion with a trailing edge respectively; and at least one of the radial vanes has an end portion which is curved for accelerating air in a substantially axial direction upon rotation of the impeller body.

In an embodiment of the present invention, there is provided a blower for use in a PAP device, including a housing, an impeller rotatably mounted in the housing, and a motor for rotating the impeller. The housing includes an inner casing and an outer casing, the impeller has a hub meridional line and a tip meridional line and at least one of the following applies: the impeller hub and/or tip meridional line(s) are within 20 degrees of perpendicular to the axis of rotation of the impeller at least at one point between an impeller inlet and an impeller outlet; at the impeller outlet the impeller hub meridian line and the impeller tip meridian line are within 20 degrees of the axis of rotation of the impeller; the impeller vanes are extended forward along the axis of rotation and also curved in the direction of rotation; the geometry of the vanes at their leading edge is chosen such that a direction of inlet flow relative to the rotating impeller blade is within 10 degrees of an angle of the vane; the leading edges of the vanes follows a curved path from a base edge of the vane to a free edge of the vane; the outer casing and impeller define an intermediate air flow path and pressurized air is bled from the flow path between the impeller and the inner casing of the blower and into contact with the motor for cooling of the motor; the housing includes bleed air channels for diverting the bled air to pass over and come into direct contact with the motor; the housing includes cooling members in thermal contact with the motor and extending into the bleed air channels for providing convective cooling of the motor.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic diagram of a prior art Positive Airway Pressure (PAP) device;

FIG. 2 is a block diagram of the controller of the prior art device shown in FIG. 1;

FIGS. 3a and 3b are front view and side view schematic representations, respectively, of a typical radial blower used in the prior art device of FIG. 1, along with the conventional cylindrical ordinates shown in bold lines;

FIG. 4 is a schematic sectional view of the prior art radial blower of FIGS. 3a and 3b illustrating the acceleration of the air in substantially radial direction only;

FIG. 5a is a front view of an impeller according to a first embodiment of the present invention;

FIG. 5b is a side view of the impeller of FIG. 5a;

FIG. 5c is a schematic sectional view of the impeller of FIG. 5a illustrating successive acceleration of the pumped air in both radial and axial direction;

FIG. 6 is a sectional view of the blower for a PAP device according to the first embodiment of the present invention, in which air is bled from the flow path through a clearance gap to cool the motor;

FIG. 7 is an exploded perspective view of the PAP device of FIG. 6 showing an arrangement of channels through which air is passed to cool the motor;

FIG. 8a is a front view of an impeller according to a second embodiment of the present invention;

FIG. 8b is a side view of the impeller of FIG. 8a;

FIG. 9 is a schematic representation of the flow geometry near the inducer portion of the impeller of FIG. 8a;

FIG. 10 is a front view of an impeller according to a third embodiment of the present invention;

FIG. 11 is an exploded view of the PAP device according to the third embodiment of the present invention;

FIG. 12 is a perspective view of the device of FIG. 11 with the housing cover and outer portion of the housing base removed;

FIG. 13 is a graph showing the typical relationship between pressure, flow rate, and rotational speed of the impeller; and, FIG. 14 is a perspective side view representing the device according to the present invention worn by a patient.

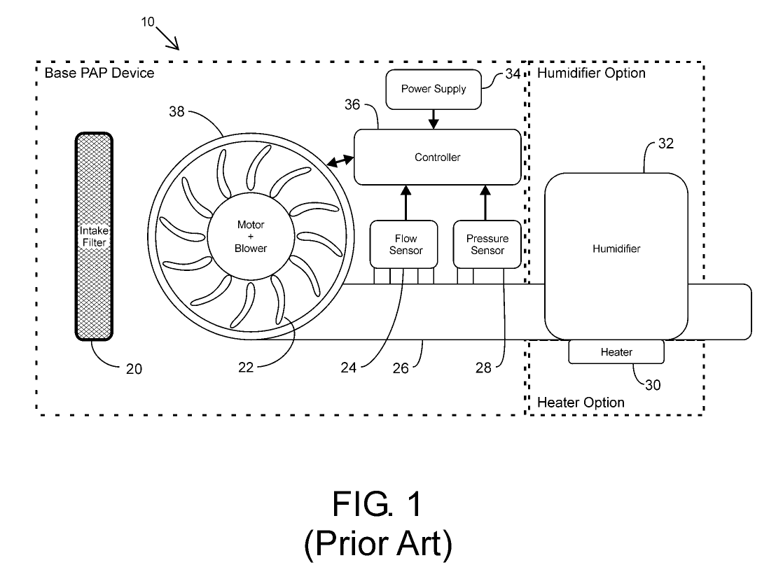

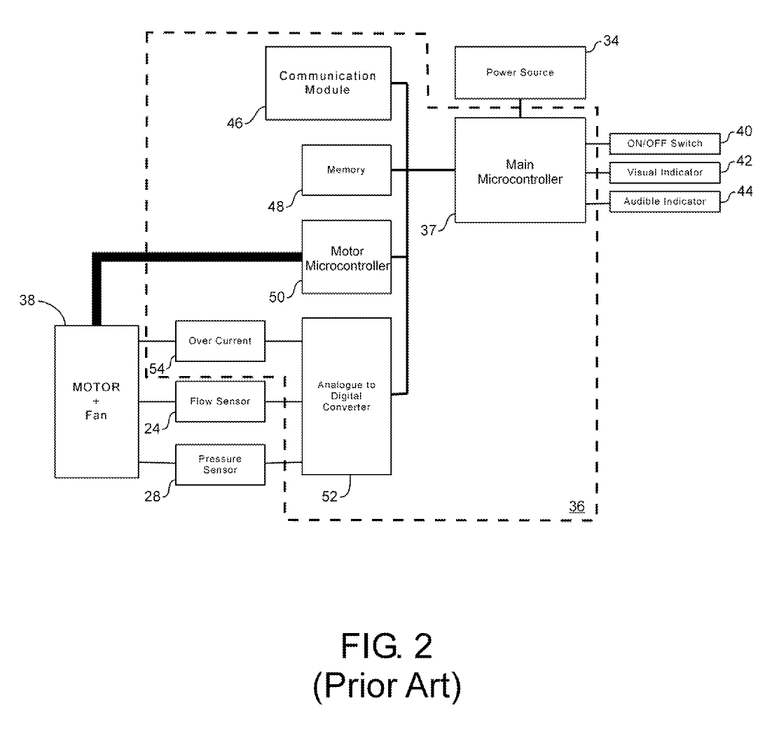

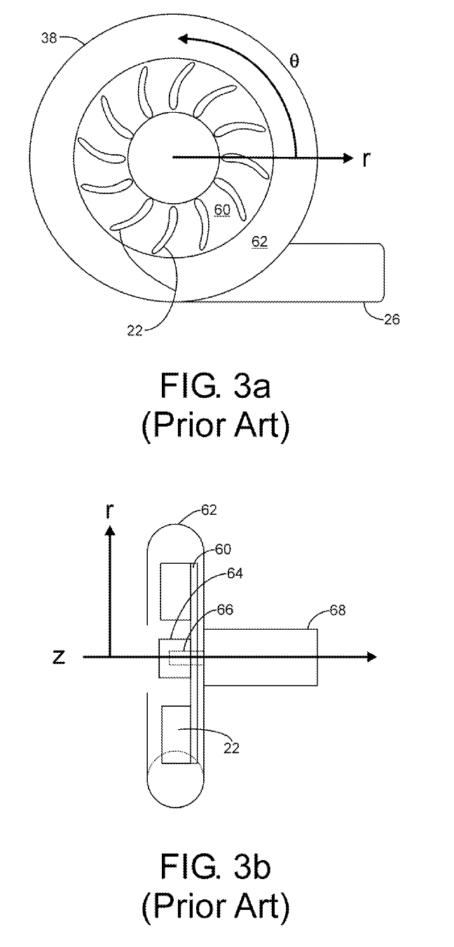

### DETAILED DESCRIPTION

Generally, the present invention provides a method for improving the air pumping efficiency of a PAP device, and a PAP device with an improved impeller that generates increased air pressure compared to prior art devices or produces at least the same air pressure at a reduced size.

As discussed earlier, the pressure developed by a rotating impeller of given geometric design is approximately proportional to the square of the velocity of the impeller at its outer periphery. Thus, the output pressure may be increased either by increasing the rotational speed of the impeller, or by increasing the impeller diameter.

In view of the move to miniaturization of wearable PAP devices and the concurrent need for reduced impeller size and a compact design, increasing the rotational speed is preferred over increasing the size of the impeller. However, the maximum rotational speed of the impeller is limited by the capacity of the motor and/or the acceptable level of noise emitted by motors and impellers operating at high rotational speeds. Thus, a blower/impeller design is needed which maximizes the use of limited available space, and motor output.

This is achieved with an air compression method and impeller design in accordance with the present invention. The output pressure of a rotary air pump in a PAP device is increased by successively accelerating air ingested by the blower in substantially radial and axial directions. Preferably, the air is first accelerated in a substantially radial direction and subsequently in substantially axial direction. Most preferably, the air is successively accelerated in a substantially axial direction, a substantially radial direction and finally again in a substantially axial direction.

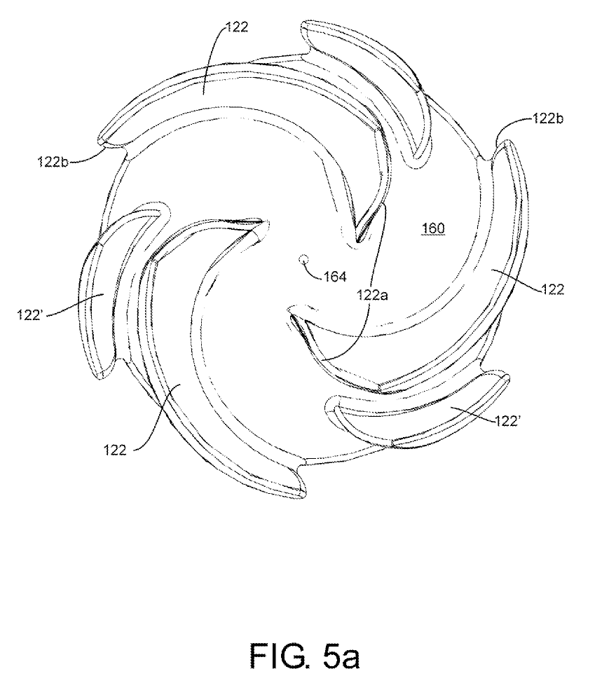

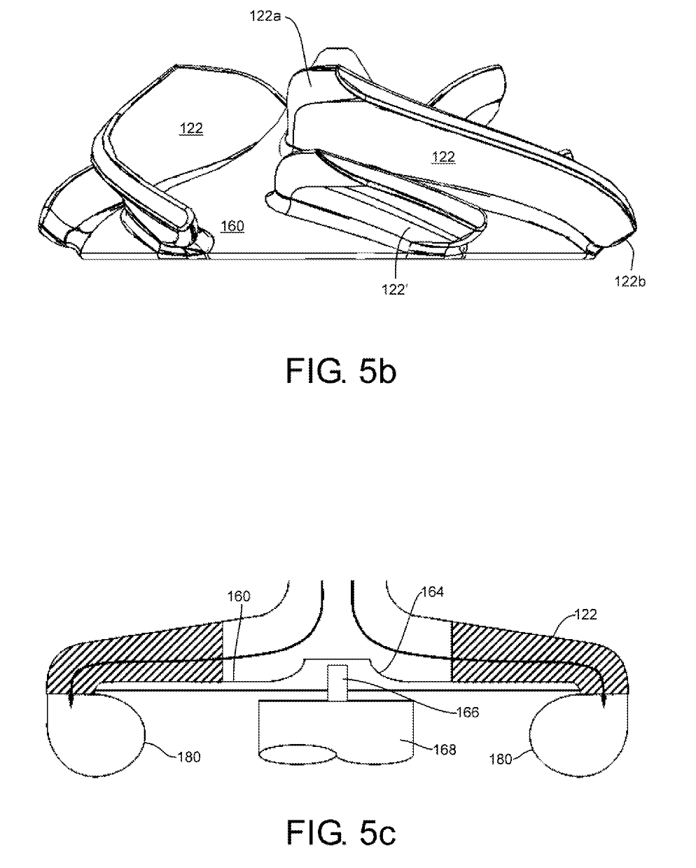

FIG. 5a shows a front view of an impeller according to a first embodiment of the present invention. The impeller 160 includes a hub 164 and a plurality of radial blades or vanes 122 and 122′ arranged in an annular array on the face of the impeller disc 160. In the illustrative example, vanes 122 extend radially outwardly from the hub 164 to the periphery of the impeller disc 160. Alternating vanes 122′ are optionally shorter in length and extend radially outwardly from the vicinity of the middle portion of the impeller disc 160 to the periphery thereof. The vanes 122 have end portions with leading edges 122a and trailing edges 122b respectively. The leading and trailing edges 122a, 122b of the vanes 122 are defined as the edges of the vanes 122 in the proximity of the hub 164 and the periphery of the impeller disc 160, respectively. The vanes 122′ have similar leading and trailing edges. FIG. 5b is a side view of the impeller shown in FIG. 5a. At least one of the end portions of the vanes is curved to accelerate air in a substantially axial direction upon rotation of the impeller. Preferably, the trailing edges 122b of the vanes 122 and the trailing edges of the vanes 122′ are curved in the axial direction for this purpose. In one embodiment, a hub meridian and a tip meridian of the impeller 160 are initially within 20 degrees of the radial direction and are also curved to within 20 degrees of the axial direction, preferably curved to be parallel to the axial direction (FIG. 5c). Although a present arrangement of vanes 122 and 122′ is shown in the example, other arrangements for the vanes 122 and 122′ are possible and will be readily apparent to a person skilled in the art.

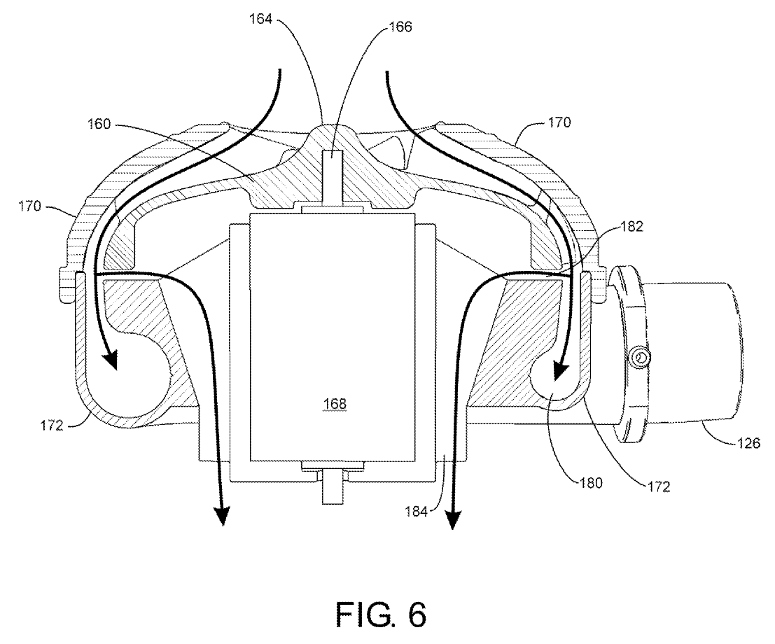

As shown in FIGS. 5c and 6, the impeller hub 164 is mounted on a shaft 166 of a motor 168. Housing cover 170 and housing base 172 are adapted to be releasably coupled to form the housing for enclosing the air as it passes through the impeller disc 160, and to guide the accelerated air toward the exit of the device through the exhaust tube 126.

Due to the geometry of the construction of the impeller 160 and curvature of the trailing edges 122b of the vanes 122 and 122′, the air drawn by the impeller 160 is successively accelerated in the radial direction, due to the rotation of the impeller, and in the axial direction by the curvature of the trailing edges 122b in the axial direction. Thus, the pressurized air exits the impeller 160 in a direction substantially parallel to the axis of rotation of the impeller 160 as shown in FIG. 5c.

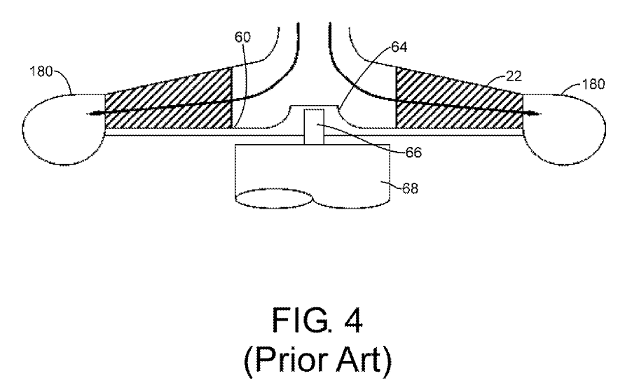

In a preferred embodiment, the overall diameter of the device is larger than the impeller 160 only by the thickness of the housing and the necessary clearance between the impeller and housing to allow rotation of the impeller without contacting the housing. With this modification, the volute 180 can be offset from the impeller 160 axially, rather than radially as in the prior art, providing a more compact design in applications where the diameter of the blower is to be minimized. The volute 180 is preferably offset in the direction toward the motor 168, as shown in FIGS. 5c and 6, making more efficient use of the space surrounding the motor 168. Using as examples the blower configurations shown in FIGS. 4 & 5c, the impeller diameter can be increased by approximately 30% according to the present invention over conventional radial impellers, while maintaining the same housing dimension. Additionally, this results in an increase in air pressure of approximately 70% at a given rotational speed.

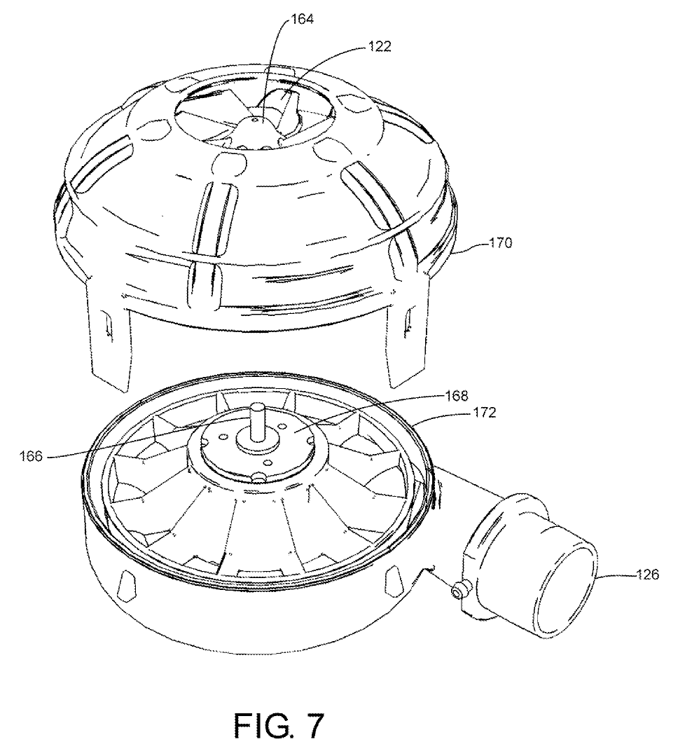

Under typical load conditions, the winding temperature of brushless DC motors may exceed 300 degrees Fahrenheit (150 degrees Celsius), and the surface temperature of the motor casing may reach temperatures between 175 and 200 degrees Fahrenheit (80 and 100 degrees Celsius). Having the motor contained in an enclosed space, and in close proximity to the patient, presents a significant safety hazard if the device overheats or catches fire. In the device according to the first embodiment of the present invention, under extreme loading conditions, the motor 168 may require as much as 3 W of cooling to remain at a safe temperature. To minimize the risk of injury, the preferred embodiment of the device includes a means of cooling the motor 168 and safely removing excess heat from the device. As shown in FIG. 6, a bleed passage 182 is provided in the clearance gap between the impeller 160 and the base 172 of the blower housing. The bleed air is diverted through channels 184 that pass over the motor 168, providing convective cooling of the motor 168, before being directed out of the device. These channels 184 preferably also contain fins, pins, or other extensions, as shown in FIG. 7, designed to facilitate heat transfer away from the motor 168, and preferably are also used as the primary means of supporting the motor 168 in the blower assembly.

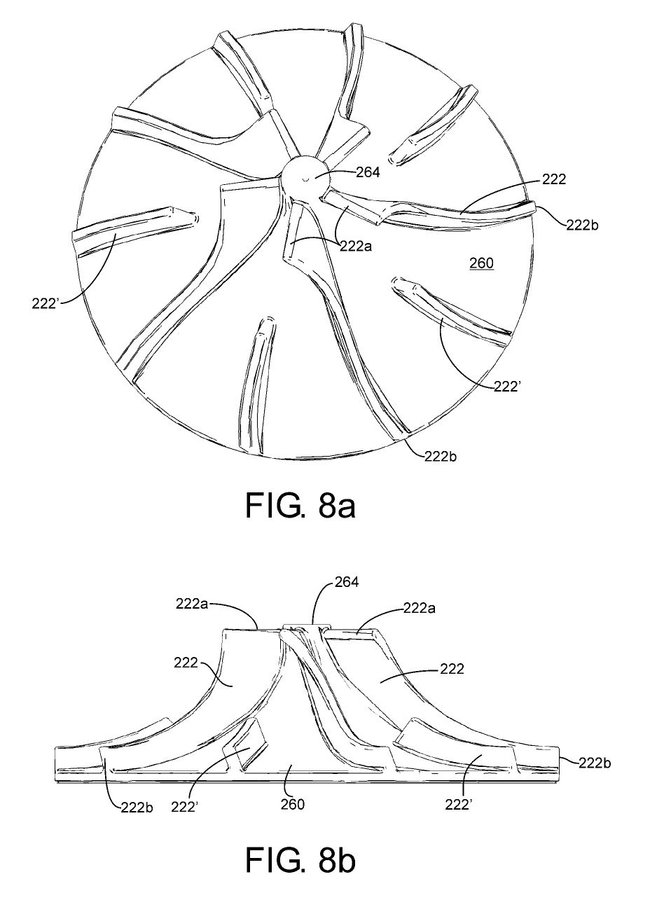

In the second embodiment of the present invention, the output pressure of a rotary air pump in a PAP device is increased by successively accelerating air ingested by the blower, first in the axial direction followed by a second acceleration in the radial direction as shown in FIGS. 8a and 8b.

The impeller 260 shown in FIG. 8a is similar to the impeller 160 shown in FIG. 5a. The impeller 260 comprises a hub 264 and a plurality of radial blades or vanes 222 and 222′ arranged in an annular array on the face of the impeller disc 260. Similar to the first embodiment, in the illustrative example, vanes 222 extend radially outwardly from the hub 264 to the periphery of the impeller disc 260. Alternating vanes 222′ optionally extend radially outwardly from the vicinity of the middle portion of the impeller disc 260 to the periphery thereof. The vanes 222 have end portions with leading edges 222a and trailing edges 222b respectively. The leading edges 222a and trailing edges 222b of the vanes 222 are defined as the edges of the vanes 222 in the proximity of the hub 264 and the periphery of the impeller disc 260, respectively. The vanes 222′ have similar leading and trailing edges. FIG. 8b is a side view of the impeller shown in FIG. 8a. Although a present arrangement of vanes 222 and 222′ is shown in the example, other arrangements for the vanes 222 and 222′ are possible and will be readily apparent to a person skilled in the art.

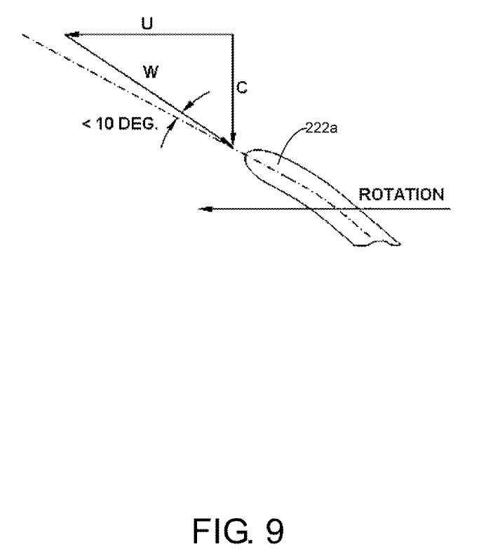

Unlike the trailing edges 122b of the vanes 122 and the trailing edges of the vanes 122′, which are curved in the axial direction, trailing edges 222b of the vanes 222 and the trailing edges of the vanes 222′ are not curved in this embodiment. However, the leading edges 222a of the vanes 222 are extended forward along the axis of rotation of the impeller 260 and are also curved in the direction of rotation as illustrated in FIGS. 8a and 8b to accelerate air in a substantially axial direction upon rotation of the impeller. As shown schematically in FIG. 9, the geometry of the vanes 222 at their leading edge 222a is chosen such that at the normal operating condition, the direction of inlet flow W relative to the rotating impeller blade, which is defined as the vector subtraction of the blade velocity U from the inlet velocity C, is within 10 degrees of the angle of the vane 222, thus acting as an air inducer. This aspect of the invention helps to draw the air entering the blower more smoothly onto the vanes 222, thereby increasing the efficiency of the blower.

In the third embodiment of the present invention, the output pressure of a rotary air pump in a PAP device is increased by successively accelerating air ingested in a substantially axial direction, a substantially radial direction and finally again in a substantially axial direction. This is achieved by combining the geometries of the vanes 122, 122′ and vanes 222 and 222′.

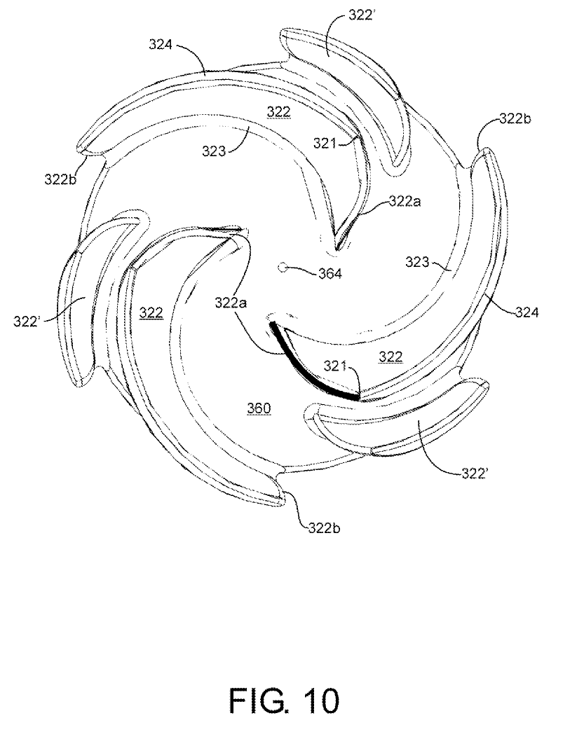

The impeller 360 shown in FIG. 10 is similar to the impeller 260 shown in FIG. 8a. The impeller 360 includes a hub 364 and a plurality of blades or vanes 322 and 322′ arranged in an annular array on the face of the impeller disc 360. Similar to the first and second embodiments, in the illustrative example, vanes 322 extend radially outwardly from the hub 364 to the periphery of the impeller disc 360. Alternating vanes 322′ optionally extend radially outwardly from the vicinity of the middle portion of the impeller disc 360 to the periphery thereof. The vanes 322 have opposite longitudinal edges, a base edge 323 at which they are connected to the impeller disc 360 and a free edge 324 spaced from the impeller disc. The vanes 322 have end portions with leading edges 322a and trailing edges 322b respectively. The leading edges 322a and trailing edges 322b of the vanes 322 are defined as the edges of the vanes 322 in the proximity of the hub 364 and the periphery of the impeller disc 360, respectively. The leading and trailing edges 322a, 322b extend between the base edge 323 and the free edge 324. The vanes 322′ have similar leading and trailing edges. Although a present arrangement of vanes 322 and 322′ is shown in the example, other arrangements for the vanes 322 and 322′ are possible and will be readily apparent to a person skilled in the art.

The curvature of the leading edges 322a of the vanes 322 and the leading edges of the vanes 322′ is similar to the curvature of the leading edges 222a of the vanes 222 of the second embodiment. In addition, the curvature of the trailing edges 322b of the vanes 322 and the trailing edges of the vanes 322′ is similar to the curvature of the trailing edges 122b of the vanes 122 and that of the trailing edges of the vanes 122′ of the first embodiment. Thus, air drawn in a direction parallel to the axis of rotation of the impeller 360 is first accelerated in the axial direction followed by a second acceleration in the radial direction, and a third acceleration in the axial direction, thereby increasing the output pressure of the rotary air pump in the PAP device.

In a preferred embodiment, the leading edges 322b of the vanes 322 and the leading edges of the vanes 322′ are also shaped such that they follow a curved path from the hub 364 of the impeller 360 to the leading tip 321 of the vanes 322 and 322′ as shown in FIG. 10 by the thickened line. The leading tip 321 is the point at which the leading edge 322a meets the free edge 324. The direction of rotation in the figure is clockwise. The path from hub 364 to tip 321 of the vanes 322 and 322′, when viewed along the axis of rotation from the front (inlet side) of the impeller 360, is preferably in the shape of a backward-leaning involute spiral. This aspect of the invention serves to diffuse the pressure wave radiating from the leading edges 322a, thus reducing the noise output of the device.

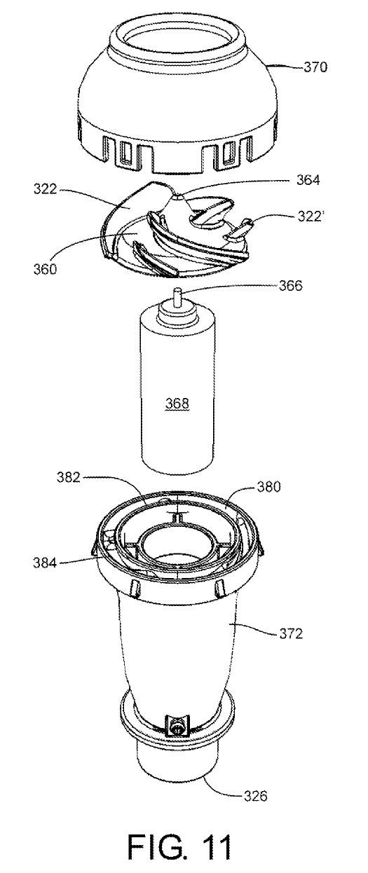

FIG. 11 shows an exploded view of the blower of a PAP device according to the third embodiment of the present invention. The blower comprises the impeller 360, motor 368 having a shaft 366, housing cover 370, housing base 372 and exhaust tube 326. The housing of the blower is designed so as to allow cooling of the motor as the air is exhausted from the blower. The housing base 372 includes an inner portion 382 and an outer portion 384. The outer portion 384 of the housing base 372 is adapted to receive and enclose the flow exiting the impeller 360. The motor 368 is adapted to be placed within the inner portion 382 of the housing base 372. It is understood that the construction shown in FIG. 11 is by of way of example only and other housing constructions are possible and will be readily apparent to those skilled in the art.

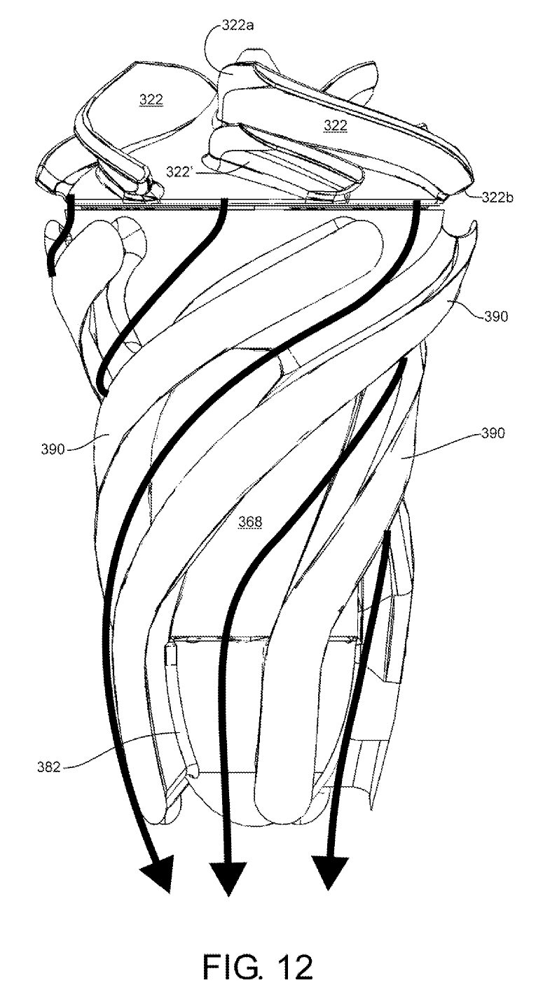

In this example, the inner portion 382 and the outer portion 384 of the housing base 372 are shaped such that they form an annular space 380 downstream of the impeller 360. This annular space, which replaces the volute or scroll 180 described in previous embodiments, allows the pressurized air to exit the blower unit in a direction parallel to the axis of rotation of the motor 368. Since this is also parallel to the direction of the inlet flow, the change in direction between the inlet and the outlet, characteristic of conventional volute designs, is eliminated, and the blower unit is more easily fitted into a compact device. As in previous embodiments, the impeller 360 contains a section where the vane meridians at the base 323 and at the free edge 324 are directed primarily in the radial direction, thus taking advantage of the centrifugal compression characteristic of radial blowers. In a preferred embodiment of the invention, the inner portion 382 of the housing base 372 of the blower is in part or in whole, comprised of the outer housing of the motor 368, whereby the patient treatment air comes into direct contact with the motor housing. In order to better illustrate this principle, FIG. 12 shows the blower of FIG. 11 in an assembled relation with the housing cover 370 and outer portion 384 of the base 372 removed.

Furthermore, the annular space 380 also contains two or more stationary protrusions 390, which are used to attach the outer portion 384 of the housing base to the inner portion of the housing base 372. Preferably, the number of stationary protrusions 390 is chosen such that it is not an integer multiple of the number of impeller vanes 322 and 322′, nor is the number of vanes 322 and 322′ an integer multiple of the number of stationary protrusions 390. This prevents more than one vane 322 or 322′ from simultaneously passing within close proximity of a stationary protrusion 390, thereby reducing the acoustic noise output by the device.

It is known in the art that higher aerodynamic efficiency is obtained in axial through-flow machines when the angular momentum of the flow, imparted by the rotating impeller, is reduced by means of a row of stator blades, placed downstream of the impeller. If properly designed, these stator blades convert a significant proportion of the angular kinetic energy of the flow into a static pressure, which would otherwise be lost if the flow were allowed to diffuse naturally. As shown in FIG. 12, the stationary protrusions 390 in the annular passage 380 are given the shape of stator blades for re-directing the substantially tangential airflow into a generally axial direction. This efficiently reduces the angular momentum of the air exiting at the exhaust tube 326.

To minimize the risk of patient injury due to burns or fire, the stationary protrusions 390 are also preferably constructed to function as cooling vanes for the motor 368. For that purpose, the protrusions 390 are in contact with both the motor 368 and the flow of air, and are constructed from a material having a low thermal resistance. Examples of suitable materials include stainless steel, aluminum alloys, and high-conductance polymer resins. This aspect of the invention serves to conduct heat from the motor 368 along the stationary protrusions 390, which are in turn cooled by the flow of air passing thereover. This allows cooling of the motor 368 to occur without the need to bleed air from the patient treatment circuit, thereby increasing the efficiency of the device, as compared to the embodiment shown in FIGS. 6 and 7. Although this leads to heating of the air supplied to the patient, the airflow rate is sufficiently large that the air temperature does not increase significantly, even under extreme operating conditions. Thus, the heating of the airflow caused by cooling of the motor does not pose an additional risk of injury.

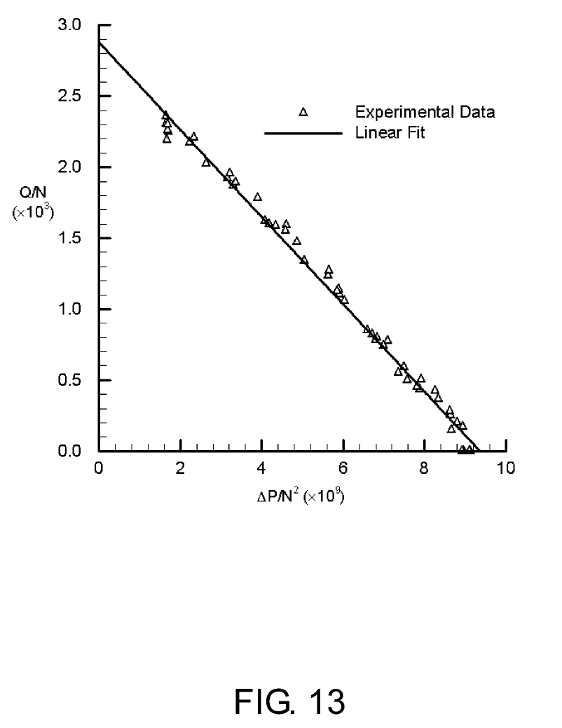

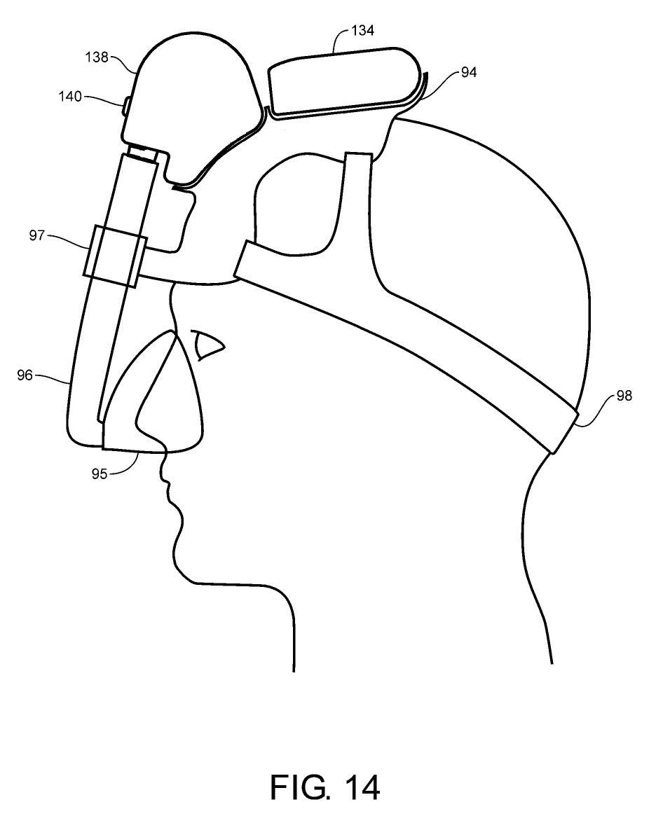

In order to diagnose certain system faults in PAP devices, accurate measurement of the flow rate through the device is required. Flow rate is often also logged for clinical purposes. Commercially available flow metering devices, for the normal range of flow rates in use, typically measure 2-3 inches in length, and up to 1 inch in diameter. Alternatives, such as flow nozzles and orifice meters, are more compact, but require an additional pressure sensor to be present. Either of these options increases the weight, size, and manufacturing cost of the PAP device. Furthermore, the pressure sensing port, or ports, in these devices can become clogged with dust or other particles, causing failure of the device, or undesired behavior due to incorrect control input. Fortunately, for a given blower, flow rate (Q) can be correlated to the motor speed (N) and output pressure (ΔP). These quantities are normally already measured in the device, since they are used to control the motor 368 and also the output pressure. The form of the correlation is typically:

$$\frac{Q}{N} = f ( \frac{Δ P}{{N}^{2}} )$$

where the function f represents a curve fit to measured data. In most cases, a low-order polynomial (e.g. quadratic), or even a straight-line fit, provides an acceptable fit to the data, as shown in the example of FIG. 13. In a preferred embodiment of the invention, the rate of volumetric flow through the device (Q) is computed from the known motor speed (N), and the measured pressure at the outlet of the device (ΔP). This eliminates the need for a dedicated flow measurement device, helping to maintain a compact design of the device.

In PAP treatment, the mask or patient interface is typically connected to the blower unit by a flexible hose, which typically measures 6 feet or more in length. In an embodiment of the present invention, shown in FIG. 14, the patient interface 95 is rigidly connected to the blower unit 138 by a coupling member 96. A suitable power supply 134 is connected to the blower unit 138. The PAP device may suitably be worn on a patient's head by means of a harness 98. The harness may include a blower and power supply mount 94 to retain the blower unit 138 and the power supply 134, and a coupling member retaining means, such as a coupling member clip 97, to retain the coupling member 96 in a desired position with respect to the patient interface 95 and the blower unit 138. The preferred PAP device has an ON/OFF control switch 140 conveniently located on the blower unit 138. The coupling member 96 may be a separate part in the assembly, or it may be formed as an extension of either the interface 95 or the blower unit 138. Alternatively, the interface 95, casing of the blower assembly 138, and coupling member 96 can be formed as a single part. The coupling member 96 is preferably designed such that the geometric relationship between the interface 95 and the blower unit 138 is adjustable to conform to the patient's need. The adjustment means may take the form of any suitable means common in the art, and preferably includes methods for providing both positional and rotational adjustment of the interface 95. Thus, the length of the coupling member 96 operatively connecting the blower unit 138 to the interface 95 is significantly reduced.

In the preferred embodiment of the invention shown in FIG. 14, the PAP device or treatment apparatus, consisting of the blower assembly 138, the patient interface 95, coupling member 96 connecting the blower assembly 138 to the patient interface 95, and harness 98 containing the components of the device, are all contained within a volume extending no further than 6 inches from the surface of the patient's head in any direction. In some embodiments the power source 134, such as a battery, may be incorporated into the blower unit 138.

In addition, for convenience of use, the device may also include a remote control unit, from which the patient may control the various settings of the unit. The remote-control unit is in wireless communication with the blower unit 138, and, at a minimum, allows the user to power the device on and off, and adjust the treatment pressure. The remote control unit also preferably allows for control of any additional accessories that may be present in the unit, including, but not limited to, humidification and heating of the treatment air. The remote control unit also comprises data acquisition, data processing, and memory storage devices that may be used to record unit performance and patient compliance, and to diagnose sleep disordered breathing events. The remote control unit preferably comprises a display screen that is used to communicate information to the patient, such as the device status, current treatment pressure, and remaining battery life, etc. Other optional display options may include the date and time, device usage, and other information as may be required for clinical purposes.

Thus, the PAP device according to the present invention is lightweight, wearable, and travel-friendly. In the present arrangement, the weight of the entire treatment apparatus, including the blower unit, power source, electronics, and patient interface, is approximately 1 lb (450 g) and is capable of producing treatment pressures typically used in PAP therapy, for example, up to 12 cm H₂O, for periods of up to 8 hours on a single battery charge. Reducing the length of coupling unit 96, fixing the geometric relationship between the blower unit 138 and the patient interface 95, and maintaining all elements of the apparatus within close proximity to each other and to the patient, significantly reduces the frequency of interface leaks due to patient movement. This is expected to significantly contribute to increased patient compliance of PAP therapy.

What is claimed is:

1. A method for increasing output pressure of a blower unit in a PAP device, the blower unit having a blower rotatable about an axis of rotation, comprising the steps of:
ingesting air into the blower unit, successively accelerating the ingested air in a radial direction substantially perpendicular to the axis of rotation and in an axial direction substantially parallel to the axis of rotation for generating compressed air;
capturing the compressed air to generate a flow of compressed air; and
exhausting the flow of compressed air from the blower unit.

2. The method of claim 1, wherein the ingested air is accelerated first in the radial direction and subsequently in the axial direction.

3. The method of claim 2, wherein the ingested air is accelerated in an axial direction substantially parallel to the axis of rotation prior to acceleration in the radial direction.

4. The method of claim 1, wherein air is ingested and exhausted in an axial direction parallel to the axis of rotation.

5. A rotary impeller for a blower unit in a PAP device, the impeller having an axis of rotation, comprising
a rotatable impeller body; and
radial vanes connected to the impeller body for accelerating air in a radial direction substantially perpendicular to the direction of rotation of the impeller body, to generate a generally radial air flow;
each radial vane having a pair of end portions which are, relative to the direction of air flow, a leading portion with a leading edge and a trailing portion with a trailing edge respectively; and at least one of the end portions of at least one of the radial vanes being curved for accelerating air in an axial direction substantially parallel to the axis of rotation, upon rotation of the impeller body.

6. The impeller of claim 5, wherein one of the trailing and leading portions of each vane is curved for accelerating air in the axial direction.

7. The impeller of claim 6, wherein each end portion of each vane is curved for accelerating air in the axial direction.

8. A blower for use in a positive airway pressure (PAP) treatment device, comprising a housing, an impeller as defined in claim 5 rotatably mounted in the housing, and a motor for rotating the impeller.

9. The blower of claim 8, wherein the housing includes an inner casing and an outer casing, the impeller has a hub meridional line and a tip meridional line and at least one of the following applies:
the impeller hub and/or tip meridional line(s) are within 20 degrees of perpendicular to the axis of rotation of the impeller at least at one point between an impeller inlet and an impeller outlet; at the impeller outlet the impeller hub meridian line and the impeller tip meridian line are within 20 degrees of the axis of rotation of the impeller;
the impeller vanes are extended forward along the axis of rotation and also curved in the direction of rotation; the geometry of the vanes at their leading edge is chosen such that a direction of inlet flow relative to the rotating impeller blade is within 10 degrees of an angle of the vane;
the leading edges of the vanes follow a curved path from a base edge of the vane to a free edge of the vane;
the outer casing and impeller define an intermediate air flow path and pressurized air is bled from the flow path between the impeller and the inner casing of the blower and into contact with the motor for cooling of the motor;
the housing includes bleed air channels for diverting the bled air to pass over and come into direct contact with the motor;
the housing includes cooling members in thermal contact with the motor and extending into the bleed air channels for providing convective cooling of the motor.

10. The blower of claim 8, wherein the housing includes an inner casing and an outer casing, and at least one of the following applies: downstream of the impeller, a space between the inner and outer casings is annular in shape, and defines a path for air directed along the axis of rotation of the impeller and motor; the annular passage further comprises two or more stationary protrusions for redirecting a rotational component of the air flow exiting the blower into an axial component; the inner casing at least partially includes an outer casing of the motor; the stationary protrusions are located between the inner and outer casings and the motor is supported in the blower primarily by said stationary protrusions; the stationary protrusions are in direct thermal contact with the motor for heat transfer away from the motor; the number of impeller blades is different from and not an integer multiple of the number of stationary protrusions; the number of stationary protrusions is not an integer multiple of the number of impeller blades.

11. The blower according to claim 10, wherein the stationary protrusions are in the shape of a cooling fin or a cooling pin for transporting heat away from the motor.

12. The blower according to claim 11, wherein the stationary protrusions are located in the flow of air directed to a user of the PAP treatment device.

13. A blower for use in a positive airway pressure treatment device comprising a housing, an impeller as defined in claim 5 rotatably mounted in the housing, a motor in the housing for rotating the impeller, the housing including an inner casing and an outer casing, and wherein a volumetric flow rate through the blower is calculated from a rotational speed of the impeller and a measured outlet pressure of the blower.

14. The blower according to claim 13, wherein the computed flow rate is used to determine system faults.

15. A positive airway pressure (PAP) treatment device for use by a patient, comprising a blower unit for producing pressurized air and having a housing, an impeller as defined in claim 5 rotatably mounted in the housing and a motor connected to the impeller for rotating the impeller, a patient interface for delivering air pressure to a patient's airway, and a coupling member connected to the blower unit for supplying pressurized air output by the blower to the patient interface.

16. The PAP treatment device of claim 15, further including a harness for supporting the blower unit on a head of the patient in an orientation in which the patient interface is properly aligned with an airway of the patient.

17. The device according to claim 15, in which the relative position between the blower assembly and the patient interface is adjustable.

18. The device according to claim 15, further comprising any of the following: a humidification system for humidifying inlet air to the blower unit, or to the pressurized air supplied to the patient interface; a heating system adapted to heat inlet air to the blower unit or the pressurized air supplied to the patient interface; a filtration system adapted to filter inlet air to the blower unit or the pressurized air supplied to the patient interface; a power source adapted to supply power to the blower unit; a power source adapted to supply power to the device, wherein the blower, the heating system, the filtration system, the humidification system, or the control circuitry, is rigidly connected to the blower unit and/or patient interface.

19. The device according to claim 15, further comprising means for wireless communication with an auxiliary control unit for use by the patient the operation of the device, preferably for powering the blower unit on and off, for increasing and decreasing the treatment pressure output by the blower, for powering on and off additional accessories, such as humidification and pre-heating of patient treatment air, modules for acquiring, processing, and storing data related to performance of the device, and/or data related to the effectiveness of treatment, the auxiliary control unit having an interface for communicating vital information to the patient/user, such as the device status, current treatment pressure, and remaining battery power.
